(19) United States
(12) Patent Application Publication
(10) Pub. No.: US 2016/0113936 A1
(43) Pub. Date: Apr. 28, 2016

(54) METHODS FOR MODULATING MONOCYTE FUNCTION

(71) Applicant: Georgia Regents Research Institute, Inc., Augusta, GA (US)

(72) Inventors: Julia Brittain, Evans, GA (US); Ciprian Anea, Grovetown, GA (US); Itia Lee, Augusta, GA (US)

(21) Appl. No.: 14/923,932

(22) Filed: Oct. 27, 2015

Related U.S. Application Data
(60) Provisional application No. 62/068,803, filed on Oct. 27, 2014.

(51) Int. Cl. A61K 31/5377; A61K 45/06

(52) U.S. Cl. CPC A61K 31/5377; A61K 45/06

(57) ABSTRACT

It has been discovered that HSP90 inhibitors can inhibit both the pro-inflammatory and pro-coagulatory potential of monocytes, in particular activated monocytes. One embodiment provides a method of inhibiting the pro-inflammatory phenotype of monocytes, preferably in human subjects, most preferably in human subjects having Sickle Cell Disease (SCD). A preferred HSP90 inhibitor is 5-(2,4-dihydroxy-5-isopropylphenyl)-N-ethyl-4-(4-(morpholinomethyl)phenyl)isoxazole-3-carboxamide (NVP-AUY922).

### CROSS-REFERENCE TO RELATED APPLICATION

This application claims priority to and benefit of U.S. Provisional Patent Application No. 62/068,803 filed on Oct. 27, 2014, and which is incorporated by reference in its entirety.

### FIELD OF THE INVENTION

The invention is generally directed to methods for modulating monocyte function for example, in the treatment of sickle cell disease.

### BACKGROUND OF THE INVENTION

It is well established that sickle cell disease (SCD) manifests global perturbations of hemostasis. Vaso-occlusion, inflammation and coagulopathy all likely contribute to the protean complications of SCD. Central to both inflammation and coagulation is the monocyte. These cells can be profoundly pro-inflammatory and can express tissue factor on their surface and thus influence both inflammation and coagulation. Monocytosis is common in SCD, as is steady state monocyte activation. Exaggerated monocytic response to stimulus may also contribute to the severity of acute events, especially acute chest syndrome.

Therefore, it is an object of the invention to provide compositions and methods for regulating or modulating the function of monocytes in a subject.

It is another embodiment to provide compositions and methods for modulating activated monocytes in subjects in need thereof.

It is still another embodiment to provide compositions and methods for treating inflammation.

It is yet another embodiment to provide methods and compositions for inhibiting or reducing coagulation.

### SUMMARY OF THE INVENTION

It has been discovered that HSP90 inhibitors can inhibit both the pro-inflammatory and pro-coagulatory potential of monocytes, in particular activated monocytes. One embodiment provides a method of inhibiting the pro-inflammatory phenotype of monocytes, preferably in human subjects, most preferably in human subjects having Sickle Cell Disease (SCD). A preferred HSP90 inhibitor is 5-(2,4-dihydroxy-5-isopropylphenyl)-N-ethyl-4-(4-(morpholinomethyl)phenyl)isoxazole-3-carboxamide (NVP-AUY922).

Another embodiment provides a method of inhibiting monocyte-induced endothelial permeability in a subject in need thereof by administering to the subject an effective amount one or more HSP90 inhibitors to reduce or inhibit monocyte-induced endothelial permeability in the subject.

Still another embodiment provides a method of treating inflammation in a subject by administering to the subject an effective amount of a HSP90 inhibitor to inhibit or reduce pro-inflammatory potential or pro-coagulatory potential or both of monocytes in the subject. Preferred subjects have SCD. In one embodiment, the effective amount of HSP90 inhibitor increases expression of HSP70 in the monocytes of the subject. The inflammation can be related to an autoimmune disease, transplant rejection, or infection.

A method for treating sickle cell disease in a subject is provided which includes administering to the subject an effective amount of a HSP90 inhibitor to inhibit or reduce pro-inflammatory potential or pro-coagulatory potential or both of monocytes in the subject.

The methods of treatment disclosed herein optionally include administering a second active agent or second therapeutic agent. The second active agent can be an anti-inflammatory agent, and agent for treating SCD, an immunosuppressant, or combinations thereof. Preferred agents for treating SCD include, but are not limited to hydroxyurea, MMF, DMF, or a combination thereof.

Pharmaceutical compositions containing a HSP90 inhibitor in combination with a second therapeutic are also provided. A preferred pharmaceutical composition contains an effective amount of NVP-AUY922 in combination with hydroxyurea, MMF, DMF or a combination thereof.

### BRIEF DESCRIPTION OF THE DRAWINGS

FIG. 1 is an autoradiograph showing HSP70 levels in monocytic THP-1 cells treated with NVP-AUY922 or 17-DMAG. β-actin levels are shown as a standard.

FIGS. 2A-2D are bar graphs showing inflammatory cytokines and tissue factor gene expression. FIG. 2A is a bar graph of Relative Tissue Factor mRNA for THP-1 cells treated with Vehicle, NVP-AUY922, 17-DMAG, TNF-α, TNF-α+NVP-AUY922, and TNF-α+17DMAG. FIG. 2B is a bar graph of Relative Tissue Factor mRNA for THP-1 cells treated with Vehicle, NVP-AUY922, 17-DMAG, LPS, and LPS+NVP-AUY922. FIG. 2C is a bar graph of TF (nM) in cells treated with vehicle, TNF-α, NVP-AUY922, TNF-α+NVP-AUY922. FIG. 2D is a bar graph of TF (nM) for cells treated with vehicle, LPS, NVP-AUY922, and LPS+NVP-AUY922.

FIG. 3 is a line graph of Increasing Permeability versus time (hrs) in primary human lung microvascular endothelial cells.

FIG. 4 is a schematic diagram of an experimental procedure for assaying HSP90 inhibitors for the treatment SCD.

FIG. 5A is a bar graph of HSP70/GAPDH (relative units) for mice treated with phosphate buffered saline (PBS) or AUY922 (25 mg/Kg B.W.) daily for four days. FIG. 5B is a bar graph of HSP70/GAPDH (relative units) for mice treated with 0.1 μg/g B.W. of LPS or 0.1 μg/g B.W. of LPS plus AUY922 (25 mg/Kg B.W.).

FIG. 6 is a bar graph showing AUY922 blocks endothelial cell activation in mice with SCD. sVCAM (pg/ml) versus mice treated with PBS control, AUY922, LPS, or LPS+AUY are shown for AA mice and SS mice.

FIG. 7 is a bar graph showing AUY-922 reduces plasma levels of TNF-alpha and Il-1-Beta in SS mice. Cytokine (ng/ml) for AA or SS mice treated with control, AUY, LPS, or LPS+AUY is shown. Cytokines are TNF-alpha and IL-1beta as indicated in the figure.

FIG. 8 is a bar graph showing treatment with AUY922 ablates the profound LPS-induced Il-6 response. The graph shows Il-6 (pg/ml) for AA mice or SS mice treated with PBS, AUY922, LPS, or LPS+AUY922.

### DETAILED DESCRIPTION OF THE INVENTION

### I. Definitions

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “treating” includes alleviating the symptoms associated with a specific disorder or condition and/or preventing or eliminating said symptoms.

The terms “reduce”, “inhibit” or “decrease” are used relative to a control. Controls are known in the art. For example a decrease response in a subject or cell treated with a compound is compared to a response in subject or cell that is not treated with the compound.

The term “pharmaceutically acceptable carrier” means a carrier combination of carrier ingredients approved by a regulatory agency of the Federal or a state government or listed in the U.S. Pharmacopoeia or other generally recognized pharmacopoeia for use in animals, mammals, and more particularly in humans. Non-limiting examples of pharmaceutically acceptable carriers include liquids, such as water and oils, including those of petroleum, animal, vegetable, or synthetic origin. Water is preferred vehicle when the compound of the invention is administered intravenously. Saline solutions and aqueous dextrose and glycerol solutions can also be employed as liquid vehicles, particularly for injectable solutions.

The term “in combination” refers to the use of more than one therapeutic agent. The use of the term “in combination” does not restrict the order in which said therapeutic agents are administered to a subject.

The term “17-AAG” refers to tanespimycin (17-N-allylamino-17-demethoxygeldanamycin), the derivative of the antibiotic geldanamycin that is an inhibitor of Hsp90.

The term “NVP-AUY922 or AUY992” refers to 5-(2,4-dihydroxy-5-isopropylphenyl)-N-ethyl-4-(4-(morpholinomethyl)phenyl)isoxazole-3-carboxamide.

### II. Methods of Treating Inflammation and Coagulation

It has been discovered that HSP90 inhibitors, in particular NVP-AUY922, can inhibit the pro-inflammatory phenotype of activated monocytes. Compositions and methods for treating pathologies associated with, related to, or induced by activated monocytes are provided. Representative diseases to be treated include, but are not limited to inflammation, SCD, transplant rejection and autoimmunity.

A. Inhibition of HSP90

Heat shock proteins (Hsp) are chaperone proteins that become up-regulated in response to cellular environmental stresses, such as elevated temperature and oxygen or nutrient deprivation. Hsp chaperones facilitate proper folding and repair of other cellular proteins, referred to as “client proteins”, and also aid the refolding of misfolded proteins. The Hsp90 family is one of the most abundant families of Hsps, representing approximately 1-2% of the total protein content in non-stressed cells and 4-6% of the protein content of cells that are stressed.

The N-terminal domain of Hsp90 contains an ATP-binding site that is central to the chaperone function. The C-terminal domain of Hsp90 mediates constitutive Hsp90 dimerization. Conformational changes of Hsp90 are orchestrated with the hydrolysis of ATP.

Hsp90 is highly conserved and facilitates folding and maturation of over 200 client proteins which are involved in a broad range of critical cellular pathways and processes. In non-stressed cells Hsp90 participates in low affinity interactions to facilitate protein folding and maturation. In stressed cells Hsp90 can assist the folding of dysregulated proteins, and is known to be involved in the development and maintenance of multiple diseases.

Hsp90 maintains the conformation and stability of many oncogenic proteins, transcription factors, steroid receptors, metalloproteases and nitric oxide synthases that are essential for survival and proliferation of cancer cells (Whitesell, et al., Nature Reviews Cancer, 5, 761-772 (2005)). Thus, Hsp90 client proteins have been associated with the development and progression of cancer. Furthermore, Hsp90 is thought to contribute to maintenance of multiple neurodegenerative diseases that are associated with protein degradation and mis-folding (proteinopathy), such as Alzheimer's disease, Huntingdon's disease and Parkinson's disease, through the mis-folding or stabilization of aberrant (neurotoxic) client-proteins.

Inhibition of Hsp90 function results in the mis-folding of client proteins, which are subsequently ubiquitinated and degraded through proteasome-dependent pathways.

Most known Hsp90 inhibitors act by binding to the N-terminus of Hsp90 and disrupting the interaction between Hsp90 and heat shock factor 1 (HSF-1). However, these Hsp90 inhibitors induce an increase in expression of Hsp70 (Bagatell, et al., Clin. Cancer Res., 6(8):3312-8 (2000)).

1. NVP-AUY922

A preferred HSP90 inhibitor is 5-(2,4-dihydroxy-5-isopropylphenyl)-N-ethyl-4-(4-(morpholinomethyl)phenyl)isoxazole-3-carboxamide (referred herein as “NVP-AUY922”). NVP-AUY922 is an experimental drug candidate for the treatment of cancer.

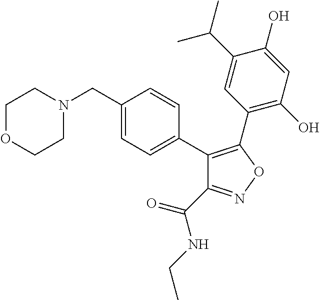 [SMILES: CCNC(=O)c1noc(-c2cc(C(C)C)c(O)cc2O)c1-c1ccc(CN2CCOCC2)cc1]

### NVP-AUY922

2. Other HSP90 Inhibitors

Additional HSP90 inhibitors that can be used in the disclosed methods and compositions include but are not limited to the following:

Ansamycins

Drug-sensitivity of Hsp90 was established using members of the natural ansamycin family of antibiotics. These antibiotics contain benzoquinone structures that confer selectivity for Hsp90 inhibition and can be used in the disclosed compositions and methods. Use of the ansamycins and derivatives as chemical probes has also been important in understanding the role of Hsp90 in stabilizing oncoproteins and how destabilizing Hsp90:client complexes leads to their cellular degradation. In some embodiments, the HSP90 inhibitor is an ansamycin.

Geldanamvcin

Geldanamycin is a naturally-occurring benzoquinone ansamycin antibiotic produced by Streptomyces hygroscopicus. Geldanamycin binds with high affinity to the N-terminal ATP binding pocket of Hsp90 and induces degradation of proteins that are mutated preferentially over their normal cellular counterparts.

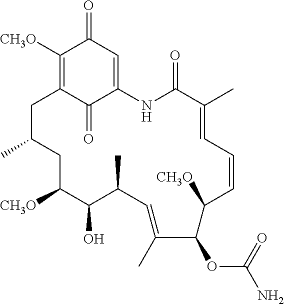

### Geldanamycin

Tanespimvcin (17-AAG)

17-allylamino-17-demethoxygeldanamycin, also known as Tanespimycin and 17-AAG, is a less toxic and more stable analog of geldanamycin. Although binding of 17-AAG to Hsp90 is weaker than that of geldanamycin, 17-AAG displays similar antitumor effects and a better toxicity profile. 17-AAG exhibits low toxicity toward non-tumor cells and has more than 100× higher affinity for Hsp90 derived from transformed cells overexpressing HER-2 (BT474, N87, SKOV3 and SKBR3) or BT474 breast carcinoma cells with 1050 values of 5-6 nM (Kamal, et al., Nature, 425:407-410 (2003); Solit, et al., Clin Cancer Res, 8:986-993 (2002)).

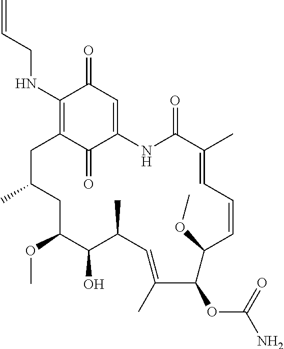 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

### Tanespimycin (17-AAG)

Alvespimvcin (17-DMAG)

17-Dimethylaminoethylamino-17-demethoxygeldanamycin (also known as Alvespimycin or 17-DMAG is a semi-synthetic derivative of Geldanamycin. 17-DMAG is the first water-soluble analog of 17-AAG and has excellent bioavailability, is widely distributed to tissues, and is quantitatively metabolized much less than is 17-AAG. 17-DMAG binds to the ATPase site of human Hsp90α with high affinity (Growth Inhibition of 50% (GI50)=51 nM for 17-DMAG vs. 120 nM for 17-AAG in the NCI 60-cell panel in vitro activity screen) (see Egorin, et al., Cancer Chemother. Pharmacol., 49:7-19 (2002) and Workman, et al., Curr Cancer Drug Targets, 3:297-300 (2003)).

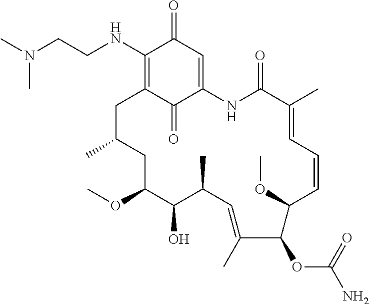 [SMILES: CO[C@H]1/C=C\C=C(/C)C(=O)NC2=CC(=O)C(NCCN(C)C)=C(C[C@@H](C)C[C@H](OC)[C@H](O)[C@@H](C)/C=C(\C)[C@@H]1OC(N)=O)C2=O]

### Alvespimycin (17-DMAG)

Retaspimvcin HCl (IPI-504)

Retaspimycin hydrochloride (also known as IPI-504) is a semi-synthetic derivative of Geldanamycin. IPI-504 is a water-soluble analog of 17-AAG that has excellent bioavailability. In the circulation, retaspimycin HCl is deprotonated and the free base hydroquinone is oxidized to 17-AAG; 17-AAG is subsequently reduced back to the hydroquinone by cellular reductase enzymes, such that the two moieties exist in a dynamic equilibrium in vivo (see Modi, et al., Breast Cancer Res., 139:107-113 (2013); Siegel, et al., Leuk. Lymphoma, 52:2308-2315 (2011)).

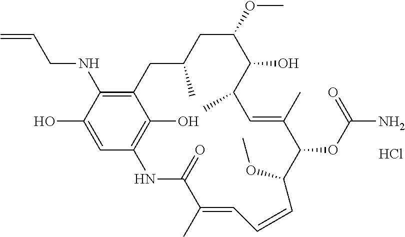 [SMILES: C=CCNc1c(O)cc2c(O)c1C[C@@H](C)C[C@H](OC)[C@H](O)[C@@H](C)/C=C(\C)[C@H](OC(N)=O)[C@@H](OC)/C=C\C=C(/C)C(=O)N2 ; Cl]

### Retaspimycin Hydrochloride (Also Known as IPI-504)

Ganetespib (STA-9090)

Ganetespib is synthetic non-geldanamycin inhibitor of Hsp90 that also binds the N-terminus ATP-binding domain. Preclinical data indicate that STA-9090 has a greater potency than 17-AAG with greater distribution throughout the tumor and no evidence of cardiac or liver toxicity (Ying, et al., Mol. Cancer Ther. 11:475-484 (2012); Shimamura, et al., Clin. Cancer Res., 18, 4973-4985 (2012)). Ganetespib exhibits sustained activity even with short exposure times; the 50% inhibitory concentrations (IC50) for Ganetespib against malignant mast cell lines are 10-50 times lower than that for 17-AAG (see Shimamura, et al., Clin. Cancer Res., 18, 4973-4985 (2012)). STA-9090 induces the overexpression of Hsp70.

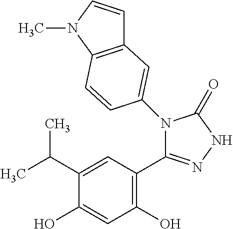 [SMILES: CC(C)c1cc(-c2n[nH]c(=O)n2-c2ccc3c(ccn3C)c2)c(O)cc1O]

### Ganetespib (STA-9090)

In addition to the geldanamycin derivatives, a series of purine scaffold inhibitors have been developed and have entered clinical trials. Many different Hsp90 inhibitors are known in the art, including C-11, SNX-2112, SNX-5542, NVP-AUY922, NVP-BEP800, CCT018159, VER-49009, PU3, BIIB021, herbimycin, derrubone, gedunin, celastrol (tripterine), (−)-epigallocatechin-3-gallate ((−)-EGCG), KW-2478, radicicol, radicicol oxime derivatives, radamide, radester, radanamycin, AT13387, debio0932, XL888 and pochonin A-F (see Hao, et al., Oncology Reports, 23:1483-92 (2010)). Diverse chemical scaffolds that have been developed as Hsp90 inhibitors are known in the art, including resorcinols, pyrimidines, aminopyrimidine, azoles and other chemotypes.

B. Diseases to be Treated

1. Inflammation

The major pro-inflammatory cytokines that are responsible for early inflammatory responses include IL1-α, IL1-β, IL-6, and TNF-α. The data provided in the Examples show that monocytic THP-1 cells treated with HSP90 inhibitors have reduced levels of Tissue Factor and inflammatory cytokine expression including, but not limited to IL1-β, IL-6, and TNF-α (See FIGS. 2A-2D and Table 1).

Therefore, one embodiment provides a method for treating inflammation in a subject in need thereof by administering an effective amount of a HSP90 inhibitor to reduce expression of one or more of IL1-β, IL-6, and TNF-α in the subject. In a preferred embodiment, the inflammation is monocyte-induced or related inflammation and the HSP90 inhibitor is NVP-AUY922. Monocyte-induced or related inflammation refers to inflammation that occurs due to the activity of monocytes in the subject. Monocyte activity includes but is not limited to the secretion of pro-inflammatory cytokines.

The inflammation can be acute inflammation or chronic inflammation. Acute inflammation refers to inflammation that has an onset in minutes or hours in response to stimuli. Chronic inflammation refers to inflammation that occurs over days. Inflammation that has not resolved itself in a day or two it can be considered chronic. Chronic inflammations can last for months or years.

2. Sickle Cell Disease

Inflammation

Mutation of the β-globin gene of hemoglobin in sickle cell anemia has pleiotropic effects in patients with sickle cell disease, including vaso-occlusion, strokes, hemolytic anemia, increased infection, and ischemic organ damage. Acute painful episodes, often called vaso-occlusive crises, are the most frequent complication of sickle disease and result in frequent hospitalizations. Monocytes of patients with sickle cell anemia are activated and can enhance vaso-occlusion through an inflammatory response promoted by the NF-kB-mediated up-regulation of adhesion molecules and tissue factor on the surfaces of endothelial cells (Belcher et al., Blood, 96(7):2451-9 (2000)).

Thus, one embodiment provides a method for inhibiting or reducing vaso-occlusion in a subject by administering to the subject an effective amount of an HSP90 inhibitor to inhibit or reduce vaso-occlusion in the subject relative to a control. In a preferred embodiment, the subject has SCD and the HSP90 inhibitor is NVP-AUY922.

Endothelial Permeability

At least 30% of the hemolysis in SCD is intravascular, which means that the endothelial wall in this disease is persistently exposed to cell-free hemoglobin. The endothelium is a semipermeable barrier that regulates the response of the vascular wall to inflammatory agonists. This response involves activation of adhesion molecule expression, increased permeability of the endothelium, and extravasations of fluid from the blood into interstitial tissue compartments. Increased vascular permeability results from opening of gaps at sites of endothelial cell-cell contacts. There are multiple indicators of systemic inflammation in SCD. Pulmonary edema and the acute chest syndrome implicate increased vascular permeability in both chronic and acute complications of SCD (Ghosh, et al., Anemia, vol. 2012, Article ID 582018, 6 pages, 2012. doi:10.1155/2012/582018.).

Thus, one embodiment provides a method for inhibiting or reducing lung endothelial permeability in a subject in need thereof, by administering to the subject an effective amount of a HSP90 inhibitor to reduce or inhibit lung endothelial permeability in the subject relative to a control. In a preferred embodiment the subject has SCD and the HSP90 inhibitor is NVP-AUY922.

Coagulation

Monocytes can provide the appropriate membrane surface for the assembly and function of all the coagulation complexes involved in tissue factor-initiated thrombin production. They can be induced to synthesize and express tissue factor antigen at their membrane surface by bacterial lipopolysaccharide (LPS) and immune complexes. In vitro, tissue factor expression by monocytes has been shown to be up-regulated by a number of (patho)physiological agonists, including, but not limited to, c-reactive protein and interleukin-1, markers of inflammation, as well as following binding to activated platelets through P-selectin via their constitutive expression of P-selectin glycoprotein ligand-1 (Bouchard, et al., Journal of Thrombosis and Haemostasis, 1: 464-469 (2003).

Thus, one embodiment provides a method for inhibiting coagulation in a subject in need thereof by administering to the subject an effective amount of a HSP90 inhibitor to inhibit or reduce expression of Tissue Factor (TF) on monocytes of the subject.

3. Autoimmune Disease

In autoimmune disease, monocytes recovered from the target organ are able to present self-antigen to autoreactive T cells. This leads to the secretion of proinflammatory cytokines, which activate monocytes further and, through the action of soluble molecules such as NO and PGE₂, curtails T cell proliferation within the target organ. In an infection, this response would favor the clearance of pathogen by monocytes and granulocytes, perhaps sparing local resources that could be consumed by T cell proliferation. In a sterile autoimmune inflammation, it serves to limit T cell expansion within the target organ, but the inflammation still causes tissue damage and harm and simply removing pro-inflammatory stimuli such as IFNγ and NO can exacerbate disease (Nicholson, et al., Current Molecular Medicine, 9, 23-29 (2009)).

One embodiment provides a method or treating or inhibiting an autoimmune reaction in a subject in need thereof by administering an effective amount of a HSP90 inhibitor to reduce or inhibit inflammation in the subject due to an autoimmune reaction. In a preferred embodiment, the HSP90 inhibitor is NVP-AUY922 and the autoimmune reaction is induced or related to activate monocyte activity.

4. Transplants

The methods and compositions described here can be used to treat or inhibit transplant rejection. The transplanted material can be cells, tissues, organs, limbs, digits or a portion of the body, preferably the human body. The transplants are typically allogenic or xenogenic. The disclosed HSP90 inhibitors are administered to a subject in an effective amount to reduce or inhibit transplant rejection. HSP90 inhibitors can be administered systemically or locally by any acceptable route of administration. In some embodiments, HSP90 inhibitors are administered to a site of transplantation prior to, at the time of, or following transplantation. In one embodiment, HSP90 inhibitors are administered to a site of transplantation parenterally, such as by subcutaneous injection.

In other embodiments, HSP90 inhibitors are administered directly to cells, tissue or organ to be transplanted ex vivo. In one embodiment, the transplant material is contacted HSP90 inhibitors prior to transplantation, after transplantation, or both.

In other embodiments, HSP90 inhibitors are administered to immune tissues or organs, such as lymph nodes or the spleen.

The transplant material can be modified prior to transplant. For example, the transplant material can be genetically modified to express a protein that aids in the inhibition or reduction of transplant rejection. In a preferred embodiment, the transplant material is genetically modified to express a heterologous nucleic acid.

The transplant material can be treated with enzymes or other materials that remove cell surface proteins, carbohydrates, or lipids that are known or suspected in being involved with immune responses such as transplant rejection.

a. Cells

Populations of any types of cells can be transplanted into a subject. The cells can be homogenous or heterogenous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mention above.

Other exemplary cells that can be transplanted include, but are not limited to, islet cells, hematopoietic cells, muscle cells, cardiac cells, neural cells, embryonic stem cells, adult stem cells, T cells, lymphocytes, dermal cells, mesoderm, endoderm, and ectoderm cells.

b. Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

c. Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of a HSP90 inhibitor to inhibit or reduce chronic transplant rejection relative to a control.

C. Co-Administration

The compositions disclosed herein can optionally include, or be co-administered with one or more additional active agents or therapeutic agents. Co-administration can include the simultaneous and/or sequential administration of the one or more additional active agents.

In one embodiment, the additional therapeutic agent is a non-steroidal anti-inflammatory drug (NSAID). Suitable NSAIDs include, but are not limited to aceclofenac, alclofenac, amfenac, aminophenazone, ampiroxicam, ampyrone, amtolmetin guacil, anitrazafen azapropazone, bendazac, benzydamine, bromfenac, bumadizone, carprofen, celecoxib, cimicoxib, clofezone, clonixin, copper ibuprofenate, COX-inhibiting nitric oxide donator, deracoxib, dexibuprofen, dexketoprofen, diclofenac, diclofenac/misoprostol, diflunisal, droxicam, epirizole, ethenzamide, etodolac, etofenamate, etoricoxib, famprofazone, felbinac, fenamic acid, fenbufen, fenclofenac, fenclozic acid, fenoprofen, feprazone, firocoxib, floctafenine, flumizole, flunixin, fluproquazone, flurbiprofen, ibuprofen, indomethacin, indometacin farnesil, indoprofen, ketoprofen, ketorolac, licofelone, lonazolac, lornoxicam, loxoprofen, lumiracoxib, magnesium salicylate, mavacoxib, mefenamic acid, meloxicam, meseclazone, miroprofen, mofebutazone, morazone, nabumetone, naproxcinod, naproxen, nepafenac, nimesulide, NOSH-aspirin, NS-398, oxaprozin, oxicam, oxyphenbutazone, parecoxib, phenazone, phenylbutazone, piroxicam, pirprofen, pranoprofen, proglumetacin, robenacoxib, rofecoxib, salicylic acid, salsalate, sulindac, suprofen, tarenflurbil, tenidap, tenoxicam, tepoxalin, tiaprofenic acid, tolfenamic acid, tolmetin, valdecoxib, vedaprofen, zomepirac, and combinations thereof.

The additional therapeutic agent can be a glucocorticoid. Representative glucocorticoids include, but are not limited to alclometasone, beclometasone dipropionate, betamethasone dipropionate, budesonide, chloroprednisone, ciclesonide, cortisol, cortisporin, cortivazol, deflazacort, dexamethasone, fludroxycortide, flunisolide, fluocinonide, fluocortolone, fluorometholone, fluticasone, fluticasone furoate, fluticasone propionate, hydrocortamate, megestrol acetate, meprednisone, methylprednisolone, mometasone furoate, otobiotic, paramethasone, prednisolone, prednisone, prednylidene, pregnadiene, pregnatriene, pregnene, proctosedyl, rimexolone, steroid dementia syndrome, tetrahydrocorticosterone, tobramycin/dexamethasone, triamcinolone, ulobetasol, and combinations thereof.

In one embodiment, the additional active agent can be an agent for treating SCD. Representative agents that can be used to treat SCD include but are not limited to hydroxyurea and fumaric esters. Examples of suitable fumaric acid esters include, but are not limited to monoethyl fumarate (MEF), monomethyl fumarate (MMF), diethyl fumarate (DEF), and dimethyl fumarate (DMF). In a preferred embodiment, the fumaric acid ester is MMF, DMF, or a combination thereof.

In another embodiment, the additional active agent is an immune suppressive agent. Exemplary immunosuppressive agents include but are not limited to prednisolone, hydrocortisone, cyclosporine, tacrolimus, azathioprine, mycophenolic acid, sirolimus, everolimus, and combinations thereof.

### III. Pharmaceutical Formulations and Administration

Compositions containing one or more HSP90 inhibitors optionally in combination with a second active agent or therapeutic agent are provided. A preferred composition contains an effective amount of one or more HSP90 inhibitors to inhibit or reduce monocyte related inflammation in a subject and an effective amount of hydroxyurea, MMF or DMF to treat SCD. The HSP90 inhibitor and the second active agent can each be in an amount of about 1 mg/kg to 10 mg/kg.

1. Parenteral Administration

In one embodiment, the compositions are administered in an aqueous solution, by parenteral injection. The formulation may also be in the form of a suspension or emulsion. In general, pharmaceutical compositions are provided including effective amounts of HSP90 inhibitors, or a derivative, analog or prodrug, or a pharmacologically active salt thereof and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as detergents and solubilizing agents (e.g., TWEEN®20, TWEEN®80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, by filtration through a bacteria retaining filter, by incorporating sterilizing agents into the compositions, by irradiating the compositions, or by heating the compositions.

2. Enteral Administration

The compositions can be formulated for oral delivery.

a. Additives for Oral Administration

Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present active compounds and derivatives. See, e.g., Remington's Pharmaceutical Sciences, 18th Ed. (1990, Mack Publishing Co., Easton, Pa. 18042) pages 1435-1712, which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the compound (or chemically modified forms thereof) and inert ingredients which protect the compound in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. HSP90 inhibitors, or a derivative, analog or prodrug, or a pharmacologically active salt thereof can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects. For oral formulations, the location of release may be the stomach, the small intestine (the duodenum, the jejunum, or the ileum), or the large intestine. Preferably, the release will avoid the deleterious effects of the stomach environment, either by protection of the active agent (or derivative) or by release of the active agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

b. Chemically Modified Forms for Oral Dosage

HSP90 inhibitors, analogs or prodrugs, thereof may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane (see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al., (1982) J. Appl. Biochem. 4:185-189).

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of HSP90 inhibitors, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be cross-linked with multivalent ions or polymers.

The matrices can be formed by solvent evaporation; spray drying, solvent extraction and other methods known to those skilled in the art. Bioerodible microspheres can be prepared using any of the methods developed for making microspheres for drug delivery, for example, as described by Mathiowitz and Langer, J. Controlled Release 5,13-22 (1987); Mathiowitz, et al., Reactive Polymers 6, 275-283 (1987); and Mathiowitz, et al., J. Appl. Polymer Sci. 35, 755-774 (1988).

The devices can be formulated for local release to treat the area that is subject to a disease, which will typically deliver a dosage that is much less than the dosage for treatment of an entire body or systemic delivery. These can be implanted or injected subcutaneously, into the muscle, fat, or swallowed.

### EXAMPLES

### Example 1

### HSP90 Inhibition Increases HSP70 Protein Levels

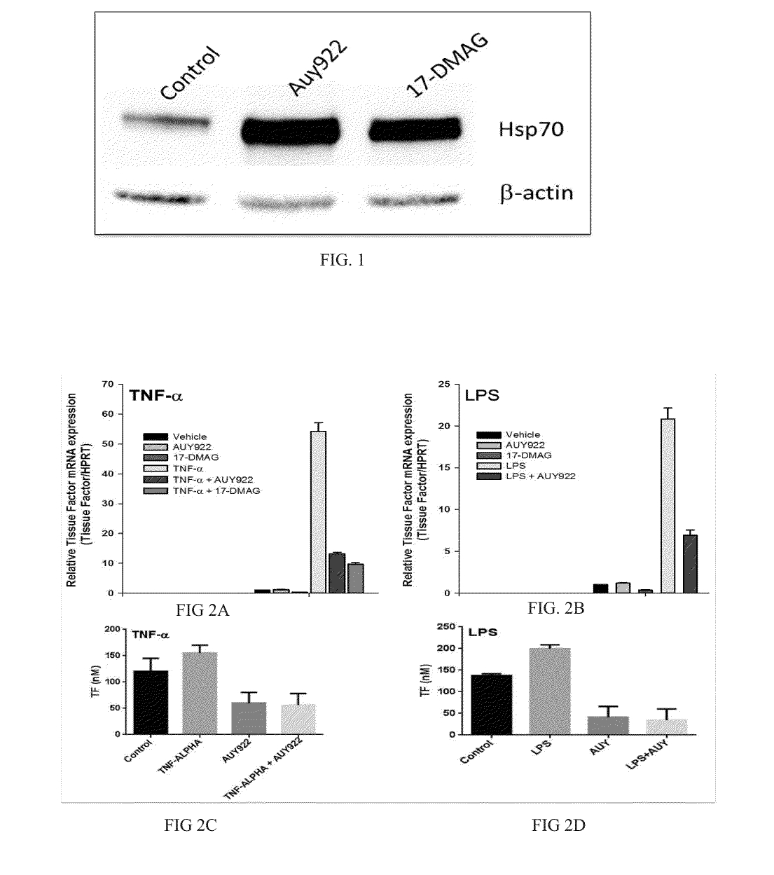

FIG. 1 shows that inhibition of HSP90 by NVP-AUY922 or 17-DMAG increases HSP70 protein levels in monocytic THP-1 cells.

### Example 2

### HSP90 Inhibition Reduces Monocyte Tissue Factor Gene Expression and Activity

Materials and Methods

Inflammatory cytokines and tissue factor gene expression were evaluated using quantitative real-time polymerase chain reaction (qRT-PCR). Amount of tissue factor was determined using a one stage clotting assay calibrated against a known amount of tissue factor (INNOVIN). See Table 1 and FIGS. 2A-2D.

Results

The data in Table 1 and FIGS. 2A-2D show that HSP90 inhibition significantly down-regulates pro-Inflammatory cytokine gene and protein expression in monocytes.

TABLE 1

| Pro- | Gene Expression (Fold) | | | | Protein Expression | | | |
|---|---|---|---|---|---|---|---|---|
| inflammatory | | | | LPS + | | | | LPS + |
| Cytokines | Control | AUY922 | LPS | AUY922 | Control | AUY922 | LPS | AUY922 |
| IL-1β | 1.0 | 0.9 | 1950 | 20 | 15.24 | 14.24 | 775.64 | 14.57 |
| IL-6 | 1.0 | 1.2 | 42.5 | 3.25 | 7.40 | 7.10 | 156.51 | 6.80 |
| IL-8 | 1.0 | 1.0 | 235.5 | 795.5 | 110.44 | 130.61 | 839.99 | 117.87 |
| TNF-α | 1.0 | 0.8 | 7.1 | 6.2 | 1611.19 | 1437.12 | 4391.73 | 1587.49 |
| IL-10 | 1.0 | 30.2 | 350.4 | 200.1 | 9.89 | 4.20 | 41.18 | 2.95 |

### Example 3

### HSP90 Inhibition Protects Against Activated Monocyte-Mediated Lung Endothelial Cell Permeability

Materials and Methods

Primary human lung microvascular endothelial cells were used in this study. Transendothelial resistance (TER): The barrier properties of cell monolayers were characterized using highly sensitive electric cell-substrate impedance sensing (ECIS) instrument and the TER data were normalized to the initial voltage.

Results

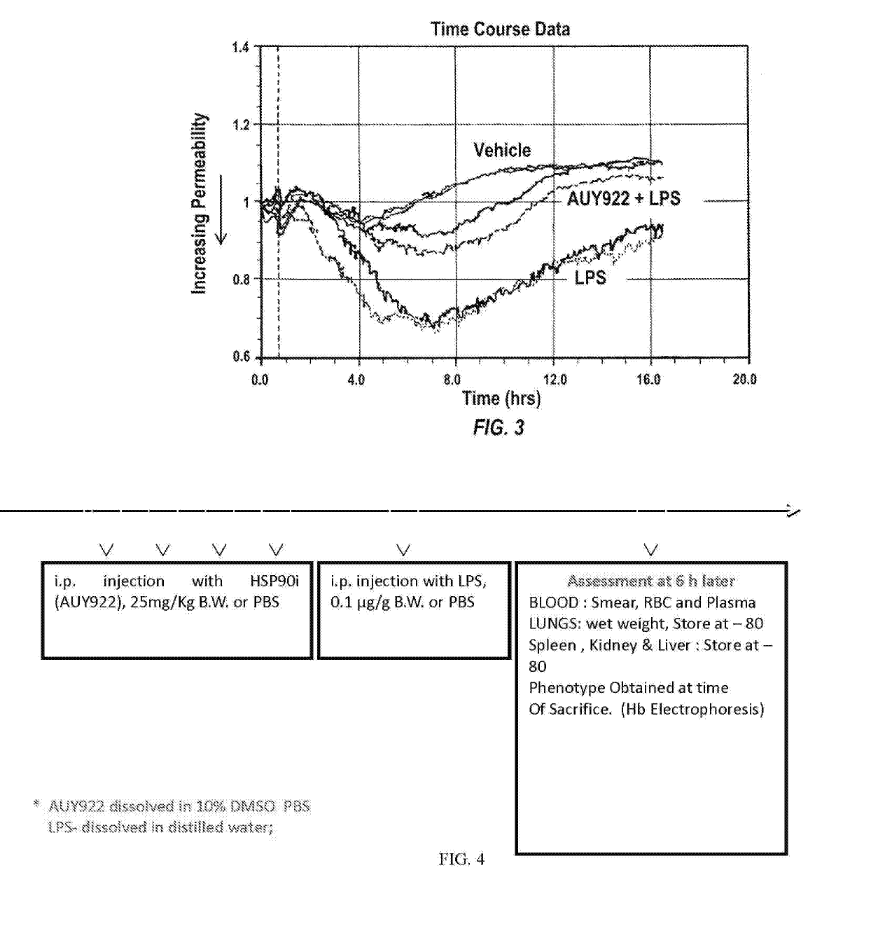

HSP90 inhibition protects against activated monocyte-mediated lung endothelial cell permeability (FIG. 3). The data suggest that HSP90 inhibition significantly reduced both pro-inflammatory and pro-coagulatory potential of monocytes from patients with SCD. The ability of HSP90 inhibitors to promote HSP70 expression, decrease inflammation and regulate the lung endothelial permeability suggests a new approach to treat SCD. These results thus position HSP90 as a potential master regulator of homeostasis and an attractive therapeutic target in patients with SCD.

### Example 4

### AUY-922 Reduces Inflammation in SS Mice

Materials and Methods

Mice

Mouse Phenotype: B6;129-Hba^{tm1(HBA)Tow} Hbb^{tm2(HBG1,HBB*)Tow}/Hbb^{tm3(HBG1,HBB)Tow}/J

### Townes Mouse Translation:

### Entire Mouse Globin Locus Removed and Replace by Human Hemoglobin S or A.

AA, SS, & SA mice are available.

Experimental Protocol

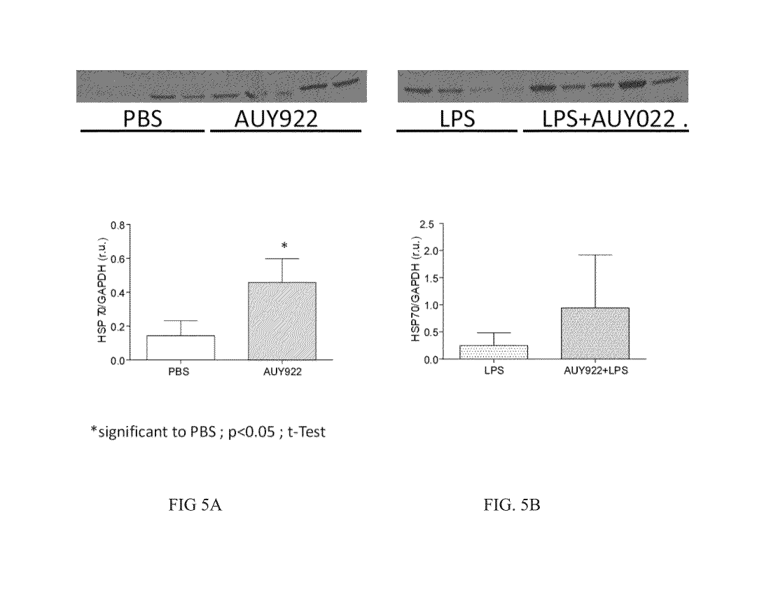

FIG. 4 shows the experimental protocol used to obtain the data in FIGS. 5-8.

Results

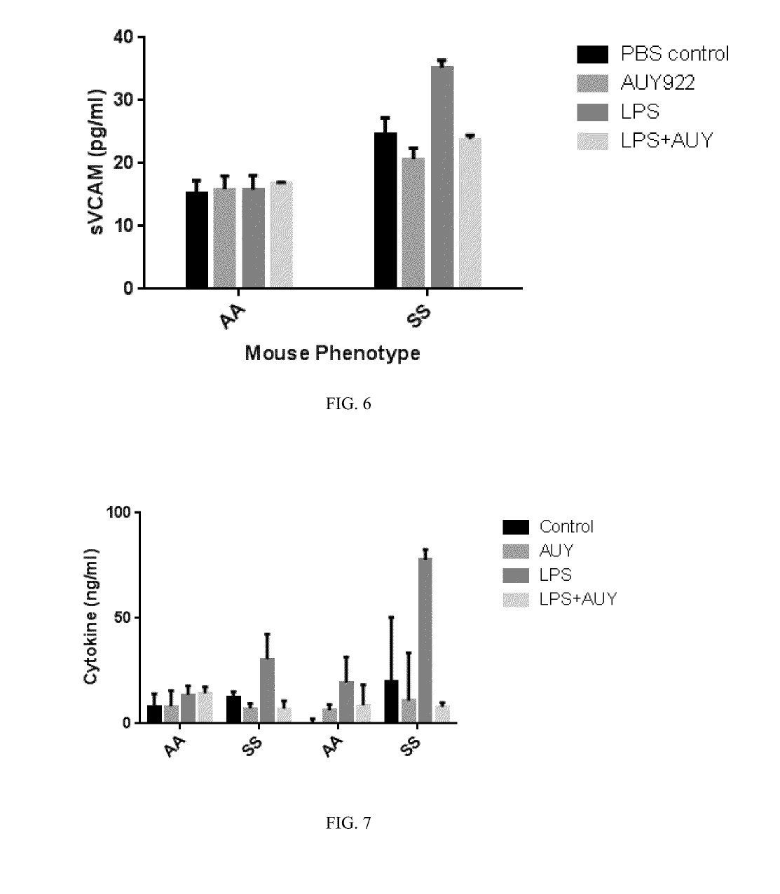

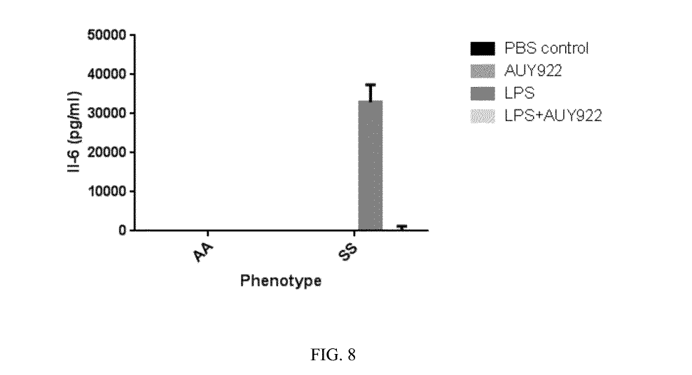

FIGS. 5A and 5B show AUY922 treatment meets pharmacodynamic requirements. FIG. 6 shows treatment with AUY922 blocks endothelial cell activation in mice with SCD. FIG. 7 shows AUY-922 reduces plasma levels of TNF-alpha and Il-1-Beta in SS mice. FIG. 8 shows treatment with AUY922 ablates the profound LPS-induced Il-6 response in mice with SCD. Taken together, the data show that treatment with AUY-922 significant decrease in inflammation in this in vivo model system for SCD.

We claim:

1. A method of treating inflammation in a subject, comprising:
administering to the subject an effective amount of a HSP90 inhibitor to inhibit or reduce pro-inflammatory potential or pro-coagulatory potential or both of monocytes in the subject.

2. The method of claim 1, wherein the subject has sickle cell disease.

3. The method of claim 1, wherein the HSP90 inhibitor increases expression of HSP70 in the monocytes of the subject.

4. The method of claim 1, wherein the HSP90 inhibitor comprises 542,4-dihydroxy-5-isopropylphenyl)-N-ethyl-4-(4-(morpholinomethyl)phenyl)isoxazole-3-carboxamide (NVP-AUY922).

5. The method of claim 1, wherein the subject is human.

6. A method for treating sickle cell disease in a subject, comprising:
administering to the subject an effective amount of a HSP90 inhibitor to inhibit or reduce pro-inflammatory potential or pro-coagulatory potential or both of monocytes in the subject.

7. The method of claim 6, wherein the HSP90 inhibitor increases expression of HSP70 in the monocytes of the subject.

8. The method of claim 6, wherein the HSP90 inhibitor comprises 542,4-dihydroxy-5-isopropylphenyl)-N-ethyl-4-(4-(morpholinomethyl)phenyl)isoxazole-3-carboxamide (NVP-AUY922).

9. The method of claim 6, wherein the subject is human.

10. The method of claim 6, further comprising administering to the subject an effective amount of second therapeutic agent.

11. The method of claim 10, wherein the second therapeutic agent is selected from the group consisting of an anti-inflammatory agent.

12. The method of claim 11, wherein the anti-inflammatory agent is selected from the group consisting of aceclofenac, alclofenac, amfenac, aminophenazone, ampiroxicam, ampyrone, amtolmetin guacil, anitrazafen azapropazone, bendazac, benzydamine, bromfenac, bumadizone, carprofen, celecoxib, cimicoxib, clofezone, clonixin, copper ibuprofenate, COX-inhibiting nitric oxide donator, deracoxib, dexibuprofen, dexketoprofen, diclofenac, diclofenac/misoprostol, diflunisal, droxicam, epirizole, ethenzamide, etodolac, etofenamate, etoricoxib, famprofazone, felbinac, fenamic acid, fenbufen, fenclofenac, fenclozic acid, fenoprofen, feprazone, firocoxib, floctafenine, flumizole, flunixin, fluproquazone, flurbiprofen, ibuprofen, indomethacin, indometacin farnesil, indoprofen, ketoprofen, ketorolac, licofelone, lonazolac, lornoxicam, loxoprofen, lumiracoxib, magnesium salicylate, mavacoxib, mefenamic acid, meloxicam, meseclazone, miroprofen, mofebutazone, morazone, nabumetone, naproxcinod, naproxen, nepafenac, nimesulide, NOSH-aspirin, NS-398, oxaprozin, oxicam, oxyphenbutazone, parecoxib, phenazone, phenylbutazone, piroxicam, pirprofen, pranoprofen, proglumetacin, robenacoxib, rofecoxib, salicylic acid, salsalate, sulindac, suprofen, tarenflurbil, tenidap, tenoxicam, tepoxalin, tiaprofenic acid, tolfenamic acid, tolmetin, valdecoxib, vedaprofen, and zomepirac.

13. A pharmaceutical composition comprising an effective amount of an HSP90 inhibitor to inhibit or reduce activated monocyte activity in a subject and an effective amount of second active agent for treating SCD.

14. The pharmaceutical composition of claim 13, wherein the HSP90 inhibitor is NVP-AUY922.

15. The pharmaceutical composition of claim 13, wherein the second active agent is hydroxyurea, MMF, DMF, or a combination thereof.

16. The pharmaceutical composition of claim 13, wherein the second active agent comprises a non-steroidal anti-inflammatory agent.

17. The pharmaceutical composition of claim 13, wherein the second active agent comprises a glucocorticoid.
